(19) United States
(12) Patent Application Publication
(10) Pub. No.: US 2023/0104881 A1
(43) Pub. Date: Apr. 6, 2023

(54) RETINOL-BASED COMPOSITION

(71) Applicant: L'OREAL, Paris (FR)

(72) Inventors: Naïma YOUSFI, Chevilly Larue (FR); Françoise HO, Chevilly Larue (FR); Majolie LIBII, Chevilly Larue (FR); Gaétane DAVID, Chevilly Larue (FR)

(21) Appl. No.: 17/905,190

(22) PCT Filed: Jun. 8, 2021
(86) PCT No.: PCT/EP2021/065310

§ 371 Date: Aug. 29, 2022

(30) Foreign Application Priority Data
Jun. 8, 2020 (FR) FR2005957

(51) Int. Cl. A61K 8/67; A61K 8/37; A61K 8/44; A61K 8/34; A61Q 1/04; A61Q 19/08; A61Q 1/10

(52) U.S. Cl. CPC A61K 8/671; A61K 8/37; A61K 8/44; A61K 8/676; A61K 8/678; A61K 8/347; A61Q 1/04; A61Q 19/08; A61Q 1/10

(57) ABSTRACT

The present invention relates to a composition, notably a cosmetic composition, in particular for making up and/or caring for keratin materials, comprising at least retinol, di-t-butyl pentaerythrityl tetrahydroxycinnamate, an ethylenediaminedisuccinic acid salt, and at least one compound chosen from ascorbic acid or an analog thereof, and/or tocopherol or a derivative thereof.

### TECHNICAL FIELD

The present invention relates to the field of caring for and/or making up keratin materials, in particular to the anti-aging care of keratin materials, notably of the skin.

For the purposes of the present invention, the term “keratin materials” notably denotes the skin, the lips and/or the eyelashes, in particular the skin and/or the lips, and preferably the skin of the body and/or the face, and more preferentially of the face.

Skin aging results from the effects of intrinsic and extrinsic factors on the skin. During the aging process, a detrimental change in the structure and functions of the skin appears. The main clinical signs due to these modifications of the skin metabolism are the appearance of wrinkles and fine lines, the cause of which is a slackening and loss of the elasticity of the tissues.

Moreover, intrinsic aging, which brings about the changes of the skin, causes in particular a slowing down of the renewal of the cells of the skin, which is reflected essentially by the appearance of detrimental clinical changes, such as the reduction in the subcutaneous adipose tissue and the appearance of small wrinkles or fine lines, and by histopathological changes, such as an increase in the number and thickness of elastic fibers, a loss of vertical fibers from the membrane of the elastic tissue, and the presence of large irregular fibroblasts in the cells of this elastic tissue.

### PRIOR ART

It is known practice to treat these signs of skin aging using cosmetic or dermatological compositions containing active agents that are capable of combating aging, of which retinoids represent a known family of active agents.

Among the compounds of the retinoid family, retinol, which is one of the forms of vitamin A, is particularly interesting. Specifically, retinol is a natural endogenous constituent of the human body. Furthermore, it is well tolerated when applied to the skin up to much higher levels than for retinoic acid.

However, when it is introduced into a cosmetic or dermatological composition for topical application, the degradation of retinol is rapid, due to the effect of light, oxygen, metal ions, oxidizing agents, water or, in particular, due to an increase in temperature.

One of the signs of retinol degradation is the change in the odor of the cosmetic compositions in which it is used.

Specifically, retinol degradation also gives rise to the release of an odor which users find particularly unpleasant and inconvenient, and this can limit its use in cosmetic compositions. Thermal degradation of retinol was notably the subject of a study published in J. Soc. Cosm. Chem. 46, 191-198 (July-August 1995).

As a result, retinol formulated in cosmetic compositions is unstable, and it has therefore already been proposed to stabilize the retinol contained in these formulations.

For example, document WO 96/07396 suggests the stabilization of retinol in a cosmetic composition in the form of an oil-in-water emulsion by using at least one fat-soluble antioxidant active agent and/or at least one sequestrant. An example of an antioxidant active agent that is mentioned is butyl hydroxytoluene (BHT). Moreover, ethylenediaminetetraacetic acid (EDTA) and derivatives thereof are mentioned as sequestrants.

However, BHT is a controversial compound, as it is potentially toxic to human beings. In addition, EDTA and BHT are neither environmentally friendly nor biodegradable. Furthermore, this system does not make it possible to solve the problem of the change in the odor of the composition.

Moreover, document WO 93/00085 describes water-in-oil emulsions comprising retinol and a stabilizing system consisting of a chelating agent, for instance EDTA, and an antioxidant. According to said document, water-in-oil emulsions containing retinol stabilized with a system consisting of a fat-soluble antioxidant and of a water-soluble antioxidant may also be prepared.

However, although said document teaches that the combination of EDTA and BHT can stabilize retinol in a composition in the form of water-in-oil emulsions, such a combination appears to be ineffective when the composition is formulated in the form of an oil-in-water emulsion.

### DISCLOSURE OF THE INVENTION

There is thus still a need for cosmetic compositions containing retinol, which are efficient in particular for combating the signs of aging of keratin materials and which are stable over time, both on storage and also during repeated use of said compositions.

There is also a need for such cosmetic compositions which are compatible with consumer requirements, in particular from an environmental point of view, and/or in particular in the light of the organoleptic properties in accordance with user expectations, such as a pleasant odor and a pleasant color.

Moreover, there is still a need for efficient stabilization of retinol in a cosmetic composition, regardless of the presentation form of the composition.

The invention is specifically directed toward meeting these needs.

### SUMMARY OF THE INVENTION

Thus, according to a first of its aspects, the present invention relates to a composition, notably a cosmetic composition, in particular for making up and/or caring for keratin materials, comprising at least:
retinol;
di-t-butyl pentaerythrityl tetrahydroxycinnamate;
an ethylenediaminedisuccinic acid salt,
at least one compound chosen from ascorbic acid or an analog thereof, and/or tocopherol or a derivative thereof.

The composition also relates to a composition, notably a cosmetic composition, in particular for making up and/or caring for keratin materials, comprising at least:
retinol;
di-t-butyl pentaerythrityl tetrahydroxycinnamate;
an ethylenediaminedisuccinic acid salt,
at least one compound chosen from ascorbic acid or an analog thereof, preferably ascorbic acid.

The composition also relates to a composition, notably a cosmetic composition, in particular for making up and/or caring for keratin materials, comprising at least:
retinol;
di-t-butyl pentaerythrityl tetrahydroxycinnamate;
an ethylenediaminedisuccinic acid salt,
at least one compound chosen from ascorbic acid or an analog thereof, preferably ascorbic acid.
at least one compound chosen from tocopherol or a derivative thereof, preferably tocopherol.

The inventors have observed, surprisingly, that the combination of at least one di-t-butyl pentaerythrityl tetrahydroxycinnamate compound, at least one ethylenediaminedisuccinic acid salt and at least one compound chosen from ascorbic acid or an analog thereof, and/or tocopherol or a derivative thereof makes it possible to efficiently stabilize the retinol contained in a cosmetic composition.

Specifically, as shown in the examples below, the compositions according to the invention are stable insofar as losses of retinol compound contained therein are low over time.

Advantageously, a composition according to the invention comprises a low content of controversial ingredients, and in particular comprises less than 0.2% by weight of butyl hydroxytoluene (BHT) and/or ethylenediaminetetraacetic acid (EDTA) or a derivative thereof, and preferably does not comprise any controversial ingredients, and in particular does not comprise either butyl hydroxytoluene (BHT) or ethylenediaminetetraacetic acid (EDTA) or a derivative thereof.

According to another of its aspects, the invention also relates to the use of at least di-t-butyl pentaerythrityl tetrahydroxycinnamate, at least one ethylenediaminedisuccinic acid salt and at least one compound chosen from ascorbic acid or an analog thereof, and/or tocopherol or a derivative thereof in a composition, in particular a cosmetic composition, containing retinol, to slow down or even prevent the degradation of the retinol compound in the composition.

A composition according to the invention is used in particular for caring for and/or making up keratin materials, and preferably for caring for keratin materials.

Thus, according to another of its aspects, the invention further relates to a cosmetic process for making up and/or caring for, preferably caring for, keratin materials, in particular the skin and/or the lips, comprising at least one step of applying a composition according to the invention to said keratin materials.

Other features, variants and advantages of the compositions according to the invention will emerge more clearly on reading the description and the examples that follow.

### DETAILED DESCRIPTION

### Retinol

As stated previously, a composition according to the invention comprises retinol, also known as vitamin A.

For the purposes of the present invention, the term “retinol” denotes all the isomers of retinol, notably all-trans retinol, 13-cis retinol, 11-cis retinol, 9-cis retinol and 3,4-didehydroretinol.

Preferably, all-trans retinol is used.

In particular, a composition according to the invention comprises an effective amount of retinol.

For the purposes of the present invention, the term “effective amount” denotes an amount of retinol which results in the desired effect via its implementation, notably the reduction in the signs of aging of the keratin materials.

Preferably, a composition according to the invention comprises at least 0.02% by weight of retinol relative to the total weight of the composition.

More particularly, a composition according to the invention may comprise an amount of retinol of between 0.02% and 5.0% by weight, notably between 0.05% and 3.0% by weight, preferably between 0.08% and 1.0% by weight, and more preferentially between 0.1% and 0.5% by weight, relative to the total weight of the composition.

It is understood that the retinol content corresponds to the content of active material, also known as the solids content, of retinol introduced into the composition.

According to a particular embodiment variant, retinol may be introduced into the composition in a form which is dissolved in an oil, such as a plant oil, for example soybean oil, notably in a content ranging from 5% to 20% by weight, preferably of about 10% by weight in the oil.

Those sold by the company BASF, notably under the name Retinol 10SU, in a content of 10% by weight of active material in soybean oil, are most particularly suitable for use.

According to another particular embodiment variant, an encapsulated form of retinol may also be used.

### Di-t-Butyl Pentaerythrityl Tetrahydroxycinnamate

A composition according to the invention also comprises at least di-t-butyl pentaerythrityl tetrahydroxycinnamate.

Di-t-butyl pentaerythrityl tetrahydroxycinnamate or tetra-di-t-butyl pentaerythrityl hydroxyhydrocinnamate is a compound which belongs to the family of cinnamic acids and derivatives thereof, the CAS number of which is 6683-19-8.

By way of example, mention may be made of the di-t-butyl pentaerythrityl tetrahydroxycinnamate compound sold by the company BASF under the name Tinogard TT®.

Preferably, a composition according to the invention comprises from 0.01% to 2.5% by weight, preferably from 0.05% to 1.5% by weight, more preferentially from 0.1% to 0.3% by weight of di-t-butyl pentaerythrityl tetrahydroxycinnamate compound, relative to the total weight of the composition.

### Ethylenediaminedisuccinic Acid Salt

A composition according to the invention also comprises at least one ethylenediaminedisuccinic acid salt.

Ethylenediaminedisuccinic acid is a compound of formula:

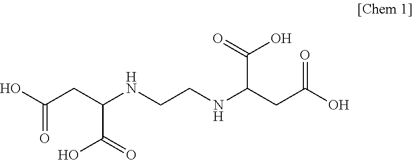 [SMILES: O=C(O)CC(NCCNC(CC(=O)O)C(=O)O)C(=O)O]

Preferably, the ethylenediaminedisuccinic acid salt is chosen from alkali metal salts, such as potassium salts and sodium salts, ammonium salts, and amine salts. Alkali metal salts of ethylenediaminedisuccinic acid are more particularly preferred.

Preferably, the ethylenediaminedisuccinic acid salt used according to the invention is trisodium ethylenediaminedisuccinate.

Such a compound is, for example, the compound sold under the name Natrlquest® E30 by the company Innospec Active Chemicals, or the compound sold under the name Octaquest E30® by the company Octel Performance Chemicals.

Preferably, a composition according to the invention comprises from 0.01% to 2.5% by weight, preferably from 0.05% to 1.5% by weight, preferably from 0.05% to 0.5% by weight, more preferentially from 0.07% to 0.3% by weight of ethylenediaminedisuccinic acid salt, relative to the total weight of the composition.

It is understood that the content of ethylenediaminedisuccinic acid salt corresponds to the content of active material, also known as the solids content, of ethylenediaminedisuccinic acid salt introduced into the composition.

According to a particular embodiment variant, the ethylenediaminedisuccinic acid salt may be introduced into the composition in a form which is dissolved in water, notably in a content ranging from 25% to 50% by weight, preferably from 35% to 40% by weight in water.

Such a compound is, for example, the compound sold under the name Natrlquest® E30 by the company Innospec Active Chemicals, at 37% by weight in water.

According to one embodiment, a composition according to the invention comprises at least one di-t-butyl pentaerythrityl tetrahydroxycinnamate compound and at least one ethylenediaminedisuccinic acid salt in a mass ratio of di-t-butyl pentaerythrityl tetrahydroxycinnamate compound/ethylenediaminedisuccinic acid salt ranging from 0.1 to 3, and preferably ranging from 0.2 to 1.5.

### Ascorbic Acid and Analogs Thereof

A composition according to the invention can also comprise at least ascorbic acid and/or an analog thereof.

Preferably the composition according to the invention comprises at least ascorbic acid and/or an analog thereof.

In one preferred embodiment, the composition according to the invention comprises ascorbic acid. By way of example, the ascorbic acid is sold by the company DSM Nutritional Products under the trade name Ascorbic Acid Fine Powder®.

Ascorbic acid, also known as vitamin C, is in particular in the L form since it can be extracted from natural products. By way of example, an ascorbic acid according to the invention is sold by the company Northeast General Pharmaceutical Factory under the trade name Ascorbic Acid EP/BP/USP/FCC/E300® or by the company CSPC Weisheng Pharmaceutical under the name Ascorbic Acid 100 Mesh®.

The analogs of ascorbic acid according to the invention are chosen from the salts, esters, ethers and sugars of ascorbic acid.

In one particular embodiment, the ascorbic acid analogs according to the invention are in the form of a monosaccharide ester of ascorbic acid or of a metal salt of phosphorylated ascorbic acid.

The monosaccharide esters of ascorbic acid that may be used in the invention are in particular glycosyl, mannosyl, fructosyl, fucosyl, galactosyl, N-acetylglucosamine and N-acetylmuramic derivatives of ascorbic acid and mixtures thereof, such as 6-O-β-D-galactopyranosyl L-ascorbic acid. The latter compounds and processes for preparing them are described in particular in documents EP-A-487 404, EP-A-425 066 and J05213736.

For its part, the metal salt of phosphorylated ascorbic acid can be chosen from ascorbyl phosphates of an alkali metal, ascorbyl phosphates of an alkaline-earth metal and ascorbyl phosphates of a transition metal, such as magnesium, sodium, potassium, calcium or zinc; such as magnesium ascorbyl phosphate.

The analogs of ascorbic acid are more particularly its salts, such as in particular sodium ascorbate, magnesium ascorbyl phosphate or sodium ascorbyl phosphate, its esters, such as in particular its acetic, propionic or palmitic esters, such as ascorbyl palmitate, or its sugars, such as in particular glycosyl ascorbic acid.

In one particular embodiment according to the invention, the ascorbic acid analogs correspond to the following formula wherein R₁ and R₂, and R₃ corresponds to a hydrogen atom and R₄ to a saturated or unsaturated, linear, optionally branched, C₁-C₁₆ alkyl group, preferably a non-branched saturated linear C₁₅ radical.

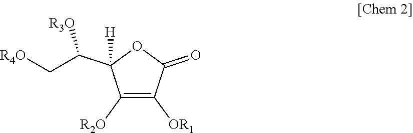

By way of example, an ascorbic acid analog may be more particularly ascorbyl palmitate (or 6-O-palmitoyl ascorbic acid) available under the trade name Ascorbyl palmitate from the company DSM Nutritional Products.

In a particular embodiment the composition of the invention comprises ascorbic acid and/or one of its analog, said analog is chosen from salts of ascorbic acid, such as sodium ascorbate, the ascorbic acid analogs chosen from Chem 2 defined above, and magnesium ascorbyl phosphate, sodium ascorbyl phosphate, and ascorbyl palmitate

In one particularly preferred embodiment according to the invention, the composition comprises ascorbic acid.

According to a particular embodiment variant, the composition according to the invention comprises from 0.025% to 1% by weight, preferably from 0.05% to 0.50% by weight, more preferentially from 0.05% to 0.3% by weight, in particular from 0.1% to 0.3% by weight, preferably from 0.1% to 0.2% by weight of ascorbic acid and/or an analog thereof, relative to the total weight of the composition.

In one particularly preferred embodiment according to the invention, the composition comprises ascorbic acid.

### Tocopherol and Derivatives Thereof

A composition according to the invention may also comprise at least tocopherol and/or a derivative thereof.

In a particular embodiment, the composition according to the invention comprises at least alpha-tocopherol.

Vitamin E is a fat-soluble vitamin covering a set of eight organic molecules, four tocopherols (alpha-, beta-, delta-, and gamma-tocopherols), and four tocotrienols (alpha-, beta-, delta-, and gamma-tocotrienols).

The tocopherols according to the invention are chosen in particular from alpha-tocopherol, beta-tocopherol, delta-tocopherol and gamma-tocopherol, or isomers thereof, and/or their mixture.

Alpha-tocopherol is preferred according to the invention, and exists in various forms: D-alpha-tocopherol, L-alpha-tocopherol and DL-alpha-tocopherol.

Particularly preferably according to the invention, the tocopherol is DL-alpha-tocopherol.

By way of example, DL-alpha-tocopherol is sold by the company DSM Nutritional Products under the trade name DL Alpha Tocopherol®.

The tocopherol derivatives according to the invention are chosen in particular from the esters, such as tocopheryl acetate, tocopheryl palmitate, tocopheryl linoleate and tocopheryl nicotinate.

According to a particular embodiment variant, the composition according to the invention comprises from 0.01% to 1% by weight, preferably from 0.05% to 0.5% by weight, more preferentially from 0.1% to 0.2% by weight of tocopherol and/or a derivative thereof, relative to the total weight of the composition.

In a preferred embodiment, the composition according to the invention comprises at least ascorbic acid and/or an analog thereof and tocopherol and/or a derivative thereof.

Preferably the composition according to the invention comprises ascorbic acid and tocopherol, in particular tocopherol is chosen from alpha-tocopherol, beta-tocopherol, delta-tocopherol, gamma-tocopherol, or isomers thereof, and/or their mixture, preferably is alpha-tocopherol.

In a particular embodiment of the invention, the composition comprises retinol, di-t-butyl pentaerythrityl tetrahydroxycinnamate, at least one ethylenediaminedisuccinic acid salt, ascorbic acid.

Preferably, a composition according to the invention comprises, in particular a cosmetic composition according to the invention comprises, retinol, di-t-butyl pentaerythrityl tetrahydroxycinnamate, at least one ethylenediaminedisuccinic acid salt, ascorbic acid, and tocopherol, preferably DL-alpha-tocopherol.

According to a preferred embodiment, a composition according to the invention, in particular a cosmetic composition according to the invention, comprises at least:
between 0.08% and 1.0% by weight of retinol,
between 0.1% and 0.3% by weight of di-t-butyl pentaerythrityl tetrahydroxycinnamate,
between 0.05% and 1.5% by weight of ethylenediaminedisuccinic acid salt,
between 0.05% and 0.50% by weight of ascorbic acid,
between 0.05% and 1% by weight of DL-alpha-tocopherol
   relative to the total weight of the composition.

### Aqueous Phase

A composition according to the invention generally comprises at least one aqueous phase and/or at least one oily phase, constituting a cosmetically acceptable medium for incorporating the effective amount of retinol, di-t-butyl pentaerythrityl tetrahydroxycinnamate and ethylenediaminedisuccinic acid salt and forming a composition, notably a cosmetic composition, according to the invention.

In particular, the aqueous phase is present in a composition according to the invention in a content ranging from 0.1% to 85% by weight, preferably from 30% to 80% by weight, and more preferentially from 50% to 80% by weight, relative to the total weight of said composition.

According to one embodiment, a composition according to the invention is anhydrous, that is to say it comprises less than 5% by weight, preferably less than 3% by weight, and more particularly less than 1% by weight of water, relative to the total weight of the composition. The aqueous phase comprises water and optionally a water-soluble solvent.

According to the present invention, the term “water-soluble solvent” denotes a compound that is liquid at room temperature and water-miscible (miscibility with water of greater than 50% by weight at 25° C. and atmospheric pressure).

The water-soluble solvents which can be used in the composition of the invention can in addition be volatile.

Among the water-soluble solvents that may be used in the composition according to the invention, mention may be made notably of lower monoalcohols containing from 1 to 5 carbon atoms such as ethanol and isopropanol, glycols containing from 2 to 8 carbon atoms such as ethylene glycol, hexylene glycol, propylene glycol, 1,3-butylene glycol and dipropylene glycol, C₃ and C₄ ketones and C₂-C₄ aldehydes.

According to an alternative embodiment, the aqueous phase of a composition according to the invention may comprise at least one C₂-C₃₂ polyol.

For the purposes of the present invention, the term “polyol” should be understood as meaning any organic molecule including at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are suitable for formulating a composition according to the present invention are in particular those notably containing from 2 to 32 carbon atoms, preferably from 3 to 16 carbon atoms.

The polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols, such as glycerol oligomers, for instance digylcerol, polyethylene glycols, and mixtures thereof.

According to a preferred embodiment of the invention, said polyol is chosen from ethylene glycol, pentaerythritol, trimethylolpropane, propylene glycol, dipropylene glycol, glycerol, polyglycerols, polyethylene glycols, and mixtures thereof.

According to a preferred mode of the invention, the composition of the invention may comprise at least one compound chosen from 1,3-propanediol, caprylyl glycol, glycerol, and mixtures thereof.

According to a preferred embodiment, the composition of the invention also comprises at least glycerol.

### Oily Phase

When the composition used according to the invention includes an oily phase, it preferably contains at least one oil, notably a cosmetic oil. It may also contain other fatty substances.

The term “oil” means a water-immiscible non-aqueous compound that is liquid at room temperature (20° C.) and at atmospheric pressure (760 mmHg).

An oily phase that is suitable for preparing the compositions, notably cosmetic compositions, according to the invention may comprise hydrocarbon-based oils, silicone oils, fluoro oils or non-fluoro oils, or mixtures thereof.

The oils may be volatile or non-volatile.

They may be of animal, plant, mineral or synthetic origin. According to an alternative embodiment, oils of silicone origin are preferred.

The term “nonvolatile” refers to an oil of which the vapor pressure at room temperature and atmospheric pressure is non-zero and is less than 10⁻³ mmHg (0.13 Pa).

For the purposes of the present invention, the term “silicone oil” means an oil comprising at least one silicon atom, and notably at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, in particular having a non-zero vapor pressure, at room temperature and atmospheric pressure, in particular having a vapor pressure ranging from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

### Volatile Oils

The volatile oils may be hydrocarbon-based oils or silicone oils.

Among the volatile hydrocarbon-based oils containing from 8 to 16 carbon atoms, mention may be made notably of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. In particular, the volatile hydrocarbon-based oil is chosen from volatile hydrocarbon-based oils containing from 8 to 16 carbon atoms and mixtures thereof.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in examples 1 and 2 of patent application WO 2008/155 059 from the company Cognis, and mixtures thereof.

Volatile silicone oils that may be mentioned include volatile linear silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane, cyclohexasiloxane and dodecamethylcyclohexasiloxane, and in particular cyclohexasiloxane.

### Nonvolatile Oils

The nonvolatile oils may notably be chosen from nonvolatile hydrocarbon-based, fluoro and/or silicone oils.

Nonvolatile hydrocarbon-based oils that may notably be mentioned include:
hydrocarbon-based oils of animal origin,
hydrocarbon-based oils of plant origin, synthetic ethers containing from 10 to 40 carbon atoms, such as dicapryl ether,
synthetic esters, for instance the oils of formula R₁COOR₂, in which R₁ represents a linear or branched fatty acid residue including from 1 to 40 carbon atoms and R₂ represents a hydrocarbon-based chain that is notably branched, containing from 1 to 40 carbon atoms, on condition that R₁+R₂ is greater than or equal to 10. The esters may be chosen especially from alcohol and fatty acid esters, for instance cetostearyl octanoate, isopropyl alcohol esters, such as isopropyl myristate, isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, and isononanoic acid esters, for instance isononyl isononanoate and isotridecyl isononanoate,
polyol esters and pentaerythritol esters, for instance dipentaerythrityl tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid, linolenic acid, and mixtures thereof,
carbonates, such as dicaprylyl carbonate,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, trimethylpentaphenyltrisiloxane, and mixtures thereof;
   and also mixtures of these various oils.

In particular, the composition may also comprise at least one nonvolatile oil, chosen in particular from nonvolatile apolar hydrocarbon-based oils, nonvolatile ester oils, and mixtures thereof.

For the purposes of the present invention, the term “apolar oil” means an oil whose solubility parameter at 25° C., δₐ, is equal to 0 (J/cm³)^{1/2}.

The definition and calculation of the solubility parameters in the Hansen three-dimensional solubility space are described in the article by C. M. Hansen: “The three dimensional solubility parameters”, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation δₐ=(δp²+δh²)^{1/2}.

The parameters δₚ, δₕ, δ_{D} and δₐ are expressed in (J/cm³P.

In particular, the nonvolatile apolar hydrocarbon-based oil is free of oxygen atoms.

Preferably, the nonvolatile apolar hydrocarbon-based oil may be chosen from linear or branched hydrocarbons of mineral or synthetic origin. In particular, it may be chosen from:
liquid paraffin or derivatives thereof,
liquid petroleum jelly,
naphthalene oil,
polybutylenes, in particular Indopol H-100 (molar mass or M_{W}=965 g/mol), Indopol H-300 (M_{W}=1340 g/mol) and Indopol H-1500 (M_{W}=2160 g/mol) sold or manufactured by the company Amoco,
polyisobutenes and hydrogenated polyisobutenes, in particular Parleam® sold by the company Nippon Oil Fats, Panalane H-300 E sold or manufactured by the company Amoco (M_{W}=1340 g/mol), Viseal 20000 sold or manufactured by the company Synteal (M_{W}=6000 g/mol) and Rewopal PIB 1000 sold or manufactured by the company Witco (M_{W}=1000 g/mol),
decene/butene copolymers and polybutene/polyisobutene copolymers, in particular Indopol L-14,
polydecenes and hydrogenated polydecenes, in particular Puresyn 10 (M_{W}=723 g/mol) and Puresyn 150 (M_{W}=9200 g/mol) sold or manufactured by the company Mobil Chemicals,
and mixtures thereof.

Said nonvolatile oil may also be an ester oil, in particular containing between 18 and 70 carbon atoms.

Examples that may be mentioned include monoesters, diesters or triesters.

The ester oils may notably be hydroxylated.

The nonvolatile ester oil may preferably be chosen from:
monoesters comprising between 18 and 40 carbon atoms in total, in particular the monoesters of formula R₁COOR₂ wherein R₁ represents a linear or branched fatty acid residue including from 4 to 40 carbon atoms and R₂ represents a hydrocarbon-based chain that is in particular branched, containing from 4 to 40 carbon atoms, on condition that R₁+R₂ is greater than or equal to 18, for instance Purcellin oil (cetostearyl octanoate), isononyl isononanoate, C₁₂ to C₁₅ alcohol benzoate, 2-ethylhexyl palmitate, octyldodecyl neopentanoate, 2-octyldodecyl stearate, 2-octyldodecyl erucate, isostearyl isostearate, diisopropyl sebacate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate or 2-diethylhexyl succinate. Preferably, they are esters of formula R₁COOR₂ wherein R₁ represents a linear or branched fatty acid residue including from 4 to 40 carbon atoms and R₂ represents a hydrocarbon-based chain that is in particular branched, containing from 4 to 40 carbon atoms, R₁ and R₂ being such that R₁+R₂ is greater than or equal to 18. Preferably, the ester comprises between 18 and 40 carbon atoms in total. Preferred monoesters that may be mentioned include isononyl isononanoate, oleyl erucate and/or 2-octyldodecyl neopentanoate;
diesters, in particular comprising between 18 and 60 carbon atoms in total, in particular between 18 and 50 carbon atoms in total. It is in particular possible to use diesters of dicarboxylic acids and of monoalcohols, preferably such as diisostearyl malate, or glycol diesters of monocarboxylic acids, such as neopentyl glycol diheptanoate or polyglyceryl-2 diisostearate, in particular such as the compound sold under the trade reference Dermol DGDIS by the company Alzo;
triesters, in particular comprising between 35 and 70 carbon atoms in total, in particular such as triesters of tricarboxylic acids, such as triisostearyl citrate, or tridecyl trimellitate, or glycol triesters of monocarboxylic acids such as polyglyceryl-2 triisostearate;
tetraesters, in particular with a total carbon number ranging from 35 to 70, such as pentaerythritol or polyglycerol tetraesters of a monocarboxylic acid, for instance pentaerythrityl tetrapelargonate, pentaerythrityl tetraisostearate, pentaerythrityl tetraisononanoate, glyceryl tris(2-decyl)tetradecanoate, polyglyceryl-2 tetraisostearate or else pentaerythrityl tetrakis(2-decyl)tetradecanoate;
polyesters obtained by condensation of unsaturated fatty acid dimer and/or trimer and of diol, such as those described in patent application FR 0 853 634, in particular such as of dilinoleic acid and of 1,4-butanediol. Mention may notably be made in this respect of the polymer sold by Biosynthis under the name Viscoplast 14436H (INCI name: dilinoleic acid/butanediol copolymer), or else copolymers of polyols and of dimer diacids, and esters thereof, such as Hailuscent ISDA;
esters and polyesters of diol dimer and of monocarboxylic or dicarboxylic acid, such as esters of diol dimer and of fatty acid and esters of diol dimer and of dicarboxylic acid dimer, in particular which may be obtained from a dicarboxylic acid dimer derived in particular from the dimerization of an unsaturated fatty acid especially of C₈ to C₃₄, especially of C₁₂ to C₂₂, in particular of C₁₆ to C₂₀ and more particularly of C₁₈, such as esters of dilinoleic diacids and of dilinoleic diol dimers, for instance those sold by the company Nippon Fine Chemical under the trade names Lusplan DD-DAS® and DD-DA7®;
vinylpyrrolidone/1-hexadecene copolymers, for instance the product sold under the name Antaron V-216 (also known as Ganex V216) by the company ISP (M_{W}=7300 g/mol);
hydrocarbon-based plant oils such as fatty acid triglycerides (which are liquid at room temperature), in particular of fatty acids containing from 7 to 40 carbon atoms, such as heptanoic or octanoic acid triglycerides or jojoba oil; mention may be made in particular of saturated triglycerides such as caprylic/capric triglyceride, glyceryl triheptanoate, glyceryl trioctanoate, and C₁₈-36 acid triglycerides such as those sold under the reference DUB TGI 24 by Stéarinerie Dubois; and unsaturated triglycerides such as castor oil, olive oil, ximenia oil and pracaxi oil;
and mixtures thereof.

The other fatty substances that may be present in the oily phase are, for example, fatty acids including from 8 to 30 carbon atoms, for instance stearic acid, lauric acid, palmitic acid and oleic acid; waxes, for instance lanolin, beeswax, carnauba wax or candelilla wax, paraffin wax, lignite wax or microcrystalline waxes, ceresin or ozokerite, and synthetic waxes, for instance polyethylene waxes and Fischer-Tropsch waxes; silicone resins such as trifluoromethyl-C₁-C₄-alkyl dimethicone and trifluoropropyl dimethicone; and silicone elastomers, for instance the products sold under the name KSG by the company Shin-Etsu, under the name Trefil or BY29 by the company Dow Corning, or under the name Gransil by the company Grant Industries.

These fatty substances may be chosen in a varied manner by a person skilled in the art in order to prepare a composition having the desired properties, for example in terms of consistency or texture.

Preferably, a composition according to the invention comprises a fatty phase containing at least one fatty substance.

According to a preferred embodiment, a composition according to the invention comprises at least one nonvolatile hydrocarbon-based oil, and preferably at least one apolar hydrocarbon-based oil.

Preferably, a composition according to the invention comprises at least two nonvolatile hydrocarbon-based oils and one nonvolatile ester oil.

According to a particularly preferred embodiment, a composition according to the invention comprises at least one nonvolatile fatty acid triglyceride, one nonvolatile carbonate oil and one nonvolatile ester oil.

According to a particularly preferred embodiment, a composition according to the invention comprises at least caprylic/capric triglyceride, dicaprylyl carbonate and diisopropyl sebacate.

Preferably, the oily phase may be present in a composition according to the invention in a content ranging from 5% to 50% by weight and preferably from 10% to 35% by weight, relative to the total weight of said composition.

### Sunscreen

A composition according to the invention may comprise one or more UV-screening agents. Thus, according to one preferred embodiment, a composition according to the invention also comprises at least one UV-screening agent.

In particular, the UV-screening agent(s) that is(are) suitable for use in the invention is(are) chosen from water-soluble UV-screening agents, liposoluble UV-screening agents, insoluble UV-screening agents, and mixtures thereof. Among these UV-screening agents, a distinction can be made between water-soluble organic screening agents, liposoluble organic screening agents, insoluble organic screening agents and inorganic screening agents.

The term “water-soluble UV-screening agent” means any compound for screening out UV radiation that can be fully dissolved or made miscible in molecular form in an aqueous phase or else that can be dissolved in colloidal form (for example in micellar form) in an aqueous phase.

The term “liposoluble UV-screening agent” means any compound for screening out UV radiation that can be fully dissolved or made miscible in molecular form in a fatty phase or else that can be dissolved in colloidal form (for example in micellar form) in a fatty phase. The term “insoluble UV-screening agent” means any compound for screening out UV radiation that has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by the company Dynamit Nobel. This solubility, determined at 70° C., is defined as the amount of product in solution in the solvent at equilibrium with an excess of solid in suspension after returning to room temperature. It may be readily evaluated in the laboratory.

The term “water-soluble organic UVA-screening agent” means any organic compound for screening out UVA radiation in the wavelength range 320 to 400 nm that can be fully dissolved or made miscible in molecular form in an aqueous phase or else that can be dissolved in colloidal form (for example in micellar form) in an aqueous phase.

By way of example, as water-soluble organic UVA-screening agents that may be used according to the present invention, mention may be made of benzene-1,4-di(3-methylidene-10-camphorsulfonic) acid (INCI name: Terephthalylidene Dicamphor Sulfonic Acid) and the various salts thereof, compounds comprising at least two benzazolyl groups bearing sulfonic groups, in particular 1,4-bis(benzimidazolyl)phenylene-3,3′,5,5′-tetrasulfonic acid (INCI name: Disodium Phenyl Dibenzimidazole Tetrasulfonate) or a salt thereof, for example sold under the name Neoheliopan AP® by the company Symrise, benzophenone compounds comprising at least one sulfonic acid function, such as benzophenone-4, benzophenone-5 or benzophenone-9.

The term “water-soluble organic UVB-screening agent” means any organic compound for screening out UVB radiation in the wavelength range ranging from 280 to 320 nm that can be fully dissolved or made miscible in molecular form in an aqueous phase or else that can be dissolved in colloidal form (for example in micellar form) in an aqueous phase.

By way of example, as water-soluble organic UVB-screening agents that may be used according to the present invention, mention may be made of water-soluble cinnamic derivatives, such as ferulic acid or 3-methoxy-4-hydroxycinnamic acid, water-soluble benzylidenecamphor compounds, water-soluble phenylbenzimidazole compounds, water-soluble p-aminobenzoic (PABA) compounds, water-soluble salicylic compounds, and mixtures thereof.

The term “liposoluble organic UVB-screening agent” means any organic compound for screening out UVB radiation in the wavelength range ranging from 280 to 320 nm that can be fully dissolved or made miscible in molecular form in a fatty phase or else that can be dissolved in colloidal form (for example in micellar form) in a fatty phase. Among the liposoluble organic UV-screening agents, some of them are liquid at room temperature.

By way of example, as liposoluble organic UV-screening agents that may be used according to the present invention, mention may be made of cinnamic derivatives, anthranilates, salicylic derivatives, dibenzoylmethane derivatives, camphor derivatives, benzophenone derivatives, β,β-diphenylacrylate derivatives, triazine derivatives, benzotriazole derivatives, benzalmalonate derivatives, in particular those cited in patent U.S. Pat. No. 5,624,663, imidazolines, p-aminobenzoic acid (PABA) derivatives, benzoxazole derivatives, as described in patent applications EP 0 832 642, EP 1 027 883, EP 1 300 137 and DE 101 62 844, screening polymers and screening silicones, such as those described in particular in patent application WO 93/04665, a-alkylstyrene-based dimers, such as those described in patent application DE 198 55 649, 4,4-diarylbutadienes as described in patent applications EP 0 967 200, DE 197 46 654, DE 197 55 649, EP-A-1 008 586, EP 1 133 980 and EP 133 981, merocyanine derivatives, merocyanines as described in patent U.S. Pat. No. 4,195,999, patent application WO 2004/006878, patent applications WO 2008/090066, WO 2011113718 and WO 2009027258, and the documents IP COM Journal No. 000179675D published on Feb. 23, 2009, IP COM Journal No. 000182396D published on Apr. 29, 2009, IP COM Journal No. 000189542D published on Nov. 12, 2009 and IP COM Journal No. IPCOM000011179D published on Mar. 4, 2004, and mixtures thereof.

Preferably, the liposoluble organic UV-screening agents may be chosen from dibenzoylmethane derivatives, such as butylmethoxydibenzoylmethane or avobenzone sold in particular under the trade name Parsol® 1789 by the company DSM Nutritional Products, salicylic derivatives, such as homosalate sold in particular under the name Eusolex® HMS by Rona/EM Industries, or ethylhexyl salicylate sold in particular under the name Neo Heliopan® OS by Symrise, β,β-diphenylacrylate derivatives, such as octocrylene sold in particular under the trade name Uvinul® N539 by BASF, or etocrylene sold in particular under the trade name Uvinul® N35 by BASF, and mixtures thereof.

By way of example, as insoluble organic UV-screening agents that may be used according to the invention, mention may be made of organic UV-screening agents of the oxalanilide type, of the triazine type, of the benzotriazole type, of the vinylamide type, of the cinnamide type, of the type comprising one or more groups which are benzazole and/or benzofuran, benzothiophene or of the indole type, of the aryl vinylene ketone type, of the phenylene bis-benzoxazinone derivative type, of the amide, sulfonamide or acrylonitrile carbamate derivative type, or mixtures thereof.

By way of example, as inorganic UV-screening agents that may be used according to the present invention, mention may be made of metal oxide pigments, such as metal oxide particles with a mean elementary particle size of less than or equal to 0.50 μm, more preferentially between 0.005 and 0.50 μm, and even more preferentially between 0.01 and 0.2 μm, better still between 0.01 and 0.1 μm and more particularly preferentially between 0.015 and 0.05 μm.

The term “elementary size” means the size of non-aggregated particles.

The sunscreen(s) may be present in a composition according to the invention in a content ranging from 1.0% to 25% by weight, preferably ranging from 3.0% to 20% by weight, relative to the total weight of the composition.

### Adjuvants

### Surfactant

According to a preferred embodiment, the composition according to the invention may also comprise at least one surfactant.

The surfactants may be chosen from nonionic, anionic, cationic and amphoteric surfactants, and mixtures thereof. Reference may be made to Kirk-Othmer's Encyclopedia of Chemical Technology, Volume 22, pages 333-432, 3rd Edition, 1979, Wiley, for the definition of the emulsifying properties and functions of surfactants, in particular pages 347-377 of this reference, for anionic, amphoteric and nonionic surfactants.

### Nonionic Surfactant

Preferably, the composition according to the invention comprises at least one nonionic surfactant.

The nonionic surfactants may in particular be chosen from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, gemini surfactants, cetyl alcohol, stearyl alcohol, and mixtures thereof.

Oxyalkylenated, in particular oxyethylenated and/or oxypropylenated, alcohols that are preferably used are those that may include from 1 to 150 oxyethylene and/or oxypropylene units, in particular containing from 20 to 100 oxyethylene units, in particular fatty alcohols, in particular of C₈-C₂₄ and preferably of C₁₂-C₁₈; those which may or may not be ethoxylated, for instance stearyl alcohol ethoxylated with 20 oxyethylene units (CTFA name Steareth-20), for instance Brij® 78 sold by the company Uniqema, cetearyl alcohol ethoxylated with 30 oxyethylene units (CTFA name Ceteareth-30), and the mixture of C₁₂-C₁₅ fatty alcohols including 7 oxyethylene units (CTFA name C₁₂₋₁₅ Pareth-7), for instance the product sold under the name Neodol 25-7® by Shell Chemicals; or in particular oxyalkylenated (oxyethylenated and/or oxypropylenated) alcohols containing from 1 to 15 oxyethylene and/or oxypropylene units, in particular ethoxylated C₅-C₂₄ and preferably C₁₂-C₁₈ fatty alcohols, such as stearyl alcohol ethoxylated with 2 oxyethylene units (CTFA name Steareth-2), for instance Brij® 72 sold by the company Uniqema.

Optionally polyoxyethylenated alkyl and polyalkyl esters of sorbitan that are preferably used include those with a number of ethylene oxide (EO) units ranging from 0 to 100. Examples that may be mentioned include sorbitan laurate 4 or 20 EO, in particular polysorbate 20 (or polyoxyethylene (20) sorbitan monolaurate) such as the product Tween® 20 sold by the company Uniqema, or else polysorbate 60, sorbitan palmitate 20 EO, sorbitan isostearate, sorbitan stearate 20 EO, sorbitan oleate 20 EO, or else the Cremophor® products (RH 40, RH 60, etc.) from BASF. The mixture of sorbitan stearate and of sucrose cocoate, sold under the name Arlacel® 2121U-FL from Croda, may also be mentioned.

Alkyl and polyalkyl glucosides or polyglucosides that are preferably used include those containing an alkyl group including from 6 to 30 carbon atoms and preferably from 6 to 18 or even from 8 to 16 carbon atoms, and containing a glucoside group preferably comprising from 1 to 5 and in particular 1, 2 or 3 glucoside units. The alkyl polyglucosides may be chosen, for example, from decyl glucoside (alkyl-C₉/C₁₁-polyglucoside (1.4)), for instance the product sold under the name Mydol 10® by the company Kao Chemicals or the product sold under the name Plantacare 2000 UP® by the company Henkel and the product sold under the name Oramix NS 10® by the company SEPPIC; caprylyl/capryl glucoside, for instance the product sold under the name Plantacare KE 3711® by the company Cognis or Oramix CG 110® by the company SEPPIC; lauryl glucoside, for instance the product sold under the name Plantacare 1200 UP® by the company Henkel or Plantaren 1200 N® by the company Henkel; coco glucoside, for instance the product sold under the name Plantacare 818 UP® by the company Henkel; caprylyl glucoside, for instance the product sold under the name Plantacare 810 UP® by the company Cognis; the mixture of arachidyl glucosyl and behenyl alcohol and arachidyl alcohol, the INCI name of which is arachidyl alcohol (and) behenyl alcohol (and) arachidyl glucoside, sold under the name Montanov® 202 by the company SEPPIC; and mixtures thereof.

### Anionic Surfactant

The anionic surfactants may be chosen from alkyl ether sulfates, carboxylates, amino acid derivatives, sulfonates, isethionates, taurates, sulfosuccinates, alkylsulfoacetates, phosphates and alkyl phosphates, polypeptides, metal salts of C₁₀-C₃₀ and especially C₁₆-C₂₅ fatty acids, in particular metal stearates and behenates, and mixtures thereof.

### Cationic Surfactant

The cationic surfactants may be chosen from alkylimidazolidiniums, such as isostearyl ethylimidonium ethosulfate, ammonium salts such as (C₁₂-C₃₀-alkyl)-tri(C₁₋₄-alkyl)ammonium halides such as N,N,N-trimethyl-1-docosanaminium chloride (or behentrimonium chloride).

### Amphoteric Surfactant

The compositions according to the invention may also contain one or more amphoteric surfactants, for instance N-acylamino acids such as N-alkyl aminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

### Silicone Surfactant

The composition may also comprise at least one silicone surfactant. By way of example, as nonionic surfactants with an HLB of greater than or equal to 8 at 25° C., used alone or as a mixture, mention may be made of dimethicone copolyol or dimethicone copolyol benzoate, and as nonionic surfactants with an HLB of less than 8 at 25° C., used alone or as a mixture, mention may be made of the cyclomethicone/dimethicone copolyol mixture.

The surfactant(s) may be present in a composition according to the invention in a proportion ranging from 0.5% to 15% by weight and preferably from 0.5% to 10% by weight, relative to the total weight of the composition.

### Gelling Agents and Thickeners

Depending on the viscosity of the composition that it is desired to obtain, one or more thickeners and/or gelling agents, which are notably hydrophilic, that is to say water-soluble or water-dispersible, may be incorporated into the composition.

According to a preferred embodiment, the gelling agent is chosen from synthetic polymeric gelling agents, in particular chosen from crosslinked acrylic homopolymers or copolymers, associative polymers, in particular associative polymers of polyurethane type, polyacrylamides, and crosslinked and/or neutralized 2-acrylamido-2-methylpropanesulfonic acid polymers and copolymers, modified or unmodified carboxyvinyl polymers, and mixtures thereof, notably as defined below.

Examples of hydrophilic gelling agents that may be mentioned include modified or unmodified carboxyvinyl polymers, such as the products sold under the names Carbopol® (CTFA name: carbomer) and Pemulen® (CTFA name: Acrylates/C₁₀₋₃₀ alkyl acrylate crosspolymer) by the company Goodrich, polyacrylamides, optionally crosslinked and/or neutralized 2-acrylamido-2-methylpropanesulfonic acid polymers and copolymers, for instance the poly(2-acrylamido-2-methylpropanesulfonic acid) sold by the company Hoechst under the name Hostacerin® AMPS (CTFA name: Ammonium polyacryldimethyltauramide), crosslinked anionic copolymers of acrylamide and of AMPS, which are in the form of a water-in-oil emulsion, such as those sold under the name Sepigel® 305 (CTFA name: Polyacrylamide/C₁₃₋₁₄ isoparaffin/laureth-7) and under the name Simulgel® 600 (CTFA name: Acrylamide/sodium acryloyldimethyltaurate copolymer/isohexadecane/polysorbate 80) by the company SEPPIC, polysaccharide biopolymers such as modified celluloses, carrageenans, gellan gum, agar-agar, xanthan gum, alginate-based compounds, in particular sodium alginate, scleroglucan gum, guar gum, inulin, pullulan, cassia gum, karaya gum, konjac gum, gum tragacanth, tara gum, acacia gum or gum arabic, and mixtures thereof.

The polymeric hydrophilic gelling agents that are suitable for use in the invention may be natural or of natural origin.

For the purposes of the invention, the term “of natural origin” denotes polymeric gelling agents obtained by modification of natural polymeric gelling agents.

These gelling agents may be particulate or non-particulate.

More specifically, these gelling agents fall within the category of polysaccharides, which may be divided into several categories.

Thus, the polysaccharides that are suitable for use in the invention may be homopolysaccharides such as fructans, glucans, galactans and mannans or heteropolysaccharides such as hemicellulose.

Similarly, they may be linear polysaccharides such as pullulan or branched polysaccharides such as gum arabic and amylopectin, or mixed polysaccharides such as starch.

More particularly, the polysaccharides that are suitable for use in the invention may be distinguished according to whether or not they are starchy. As representatives of the starchy polysaccharides, mention may be made most particularly of native starches, modified starches, and particulate starches.

In general, the non-starchy polysaccharides may be chosen from polysaccharides produced by microorganisms; polysaccharides isolated from algae, and higher plant polysaccharides, such as homogeneous polysaccharides, in particular celluloses and derivatives thereof, such as hydroxyethylcellulose, or fructosans, heterogeneous polysaccharides such as gum arabics, galactomannans, glucomannans and pectins, and derivatives thereof; and mixtures thereof. The thickener(s) and/or gelling agent(s) may be present in a composition according to the invention in a content ranging from 0.05% to 5.0% by weight, preferably from 0.1% to 4.0%, relative to the total weight of the composition.

### Fillers

A composition according to the invention may also comprise at least one filler.

The filler may be chosen from pigments, titanium oxide, red iron oxide, yellow iron oxide, black iron oxide, boron nitride, nacres, synthetic or natural mica, nacres comprising mica and titanium oxide, silica powder, talc, polyamide particles and notably those sold under the name Orgasol® by the company Atochem, polyethylene powders, microspheres based on acrylic copolymers, such as those made of ethylene glycol dimethacrylate/lauryl methacrylate copolymer sold by the company Dow Corning under the name Polytrap®, expanded powders such as hollow microspheres and notably the microspheres sold under the name Expancel® by the company Kemanord Plast or under the name Micropearl® F 80 ED by the company Matsumoto, silicone resin microbeads such as those sold under the name Tospearl® by the company Toshiba Silicone, and mixtures thereof.

Preferably, the composition according to the invention comprises boron nitride.

These fillers may be present in a composition according to the invention in a content ranging from 0.1% to 5.0% by weight, preferably from 1.0% to 3.0% by weight, relative to the total weight of the composition.

### Active Agents

A composition according to the invention may comprise additional active agents, in particular anti-aging active agents other than the retinol used according to the invention.

By way of example of anti-aging active agents, mention may be made of sodium hyaluronate, n-octanoyl-5-salicylic acid, adenosine, c-beta-d-xylopyranoside-2-hydroxypropane and the sodium salt of (3-hydroxy-2-pentylcyclopentyl)acetic acid.

Such active agents may be present in a composition according to the invention in a content ranging from 0.05% to 5.0% by weight, preferably from 0.05% to 1.5% by weight, relative to the total weight of the composition.

As mentioned above, a composition according to the invention is preferably free of compounds which may be harmful to human beings and/or the environment, i.e. it comprises less than 0.2% by weight, in particular less than 0.1% by weight, preferably less than 0.05% by weight, and more preferentially less than 0.01% by weight, or even is totally free of compounds which may be harmful to human beings and/or the environment, in particular free of butyl hydroxytoluene (BHT) and/or ethylenediaminetetraacetic acid (EDTA) or a salt thereof.

Thus, a composition according to the invention is in particular free of butyl hydroxytoluene (BHT) and/or ethylenediaminetetraacetic acid (EDTA) or a salt thereof, and preferably is totally free of ethylenediaminetetraacetic acid or a salt thereof.

A composition according to the invention may also include at least one additive chosen from the adjuvants conventionally used in the cosmetic field, such as preserving agents, fragrances, colorants, polar additives, film-forming polymers, pH modifiers (acids or bases), cosmetic active agents, for instance moisturizers, cicatrizing agents, agents for combating greasy skin, and/or anti-pollution agents, and mixtures thereof.

Needless to say, a person skilled in the art will take care to select this or these optional additional compound(s), and/or the amount thereof, such that the advantageous properties of a composition according to the invention are not, or are not substantially, adversely affected by the envisioned addition.

### Composition

As stated previously, a composition according to the invention may be cosmetic and/or dermatological, and is preferably cosmetic.

A composition according to the invention is generally suitable for topical application to the skin and thus generally comprises a physiologically acceptable medium, i.e. a medium that is compatible with the skin.

It is preferably a cosmetically acceptable medium, i.e. a medium which has a pleasant color, odor and feel and which does not cause any unacceptable discomfort, i.e. stinging, tautness or redness, liable to discourage the user from applying this composition.

A cosmetic composition according to the invention may be in any presentation form conventionally used in the cosmetic field for the intended applications, in particular for topical application.

For topical application to keratin materials, and in particular the skin or its integuments, a composition may in particular be in the form of an aqueous or oily solution or of a dispersion of the lotion or serum type, of emulsions of liquid or semi-liquid consistency of the milk type, obtained by dispersing a fatty phase in an aqueous phase (oil-in-water) or conversely (water-in-oil), or of suspensions or emulsions of soft consistency of the aqueous or anhydrous gel or cream type, or else of microcapsules or microparticles, or of vesicular dispersions of ionic and/or nonionic type. These compositions are prepared according to the usual methods.

Preferably, a composition according to the invention is in the form of an oil-in-water (O/W) emulsion, an emulsified gel or an oily solution.

In particular, a composition according to the invention has a pH ranging from 3 to 8. Preferably, the pH of the composition ranges from 4 to 7 and more preferentially from 5 to 7.

A composition according to the invention may be prepared according to the techniques that are well known to those skilled in the art.

### Intended Use of the Composition

A composition according to the invention may be in the form of a cosmetic composition for caring for and/or making up keratin materials, in particular of the body or of the face, preferably of the face.

These compositions may constitute cleansing, protective, treating or care creams for the face, the hands or the body, for example day creams, night creams, makeup creams, foundation creams, antisun creams, fluid foundations, protective or care body milks, antisun milks, lotions, or blister treatment products.

Thus, a composition according to the invention may be used as a care product and/or antisun product for the face and/or body, with a liquid to semi-liquid consistency, such as oils, milks, lotions, more or less smooth creams, or gel-creams.

Preferably, a composition according to the invention is in the form of a cosmetic composition for caring for keratin materials, in particular the skin of the body or of the face, preferably of the face.

In particular, a composition of the invention may be in the form of an anti-aging care composition for the skin of the body or of the face, in particular of the face.

According to another embodiment, a composition of the invention may be in the form of a composition for making up keratin materials, in particular of the body or of the face, preferably of the face.

Thus, according to a submode of this embodiment, a composition of the invention may be in the form of a makeup base composition for making up. A composition of the invention may in particular be in the form of a foundation.

According to yet another submode of this embodiment, a composition of the invention may be in the form of a lip product, notably a lipstick.

According to yet another submode of this embodiment, a composition of the invention may be in the form of a product for the eyebrows, in particular an eyebrow pencil.

Such compositions are in particular prepared according to the general knowledge of those skilled in the art.

Thus, the invention also relates to the use of a composition according to the invention for caring for and/or making up keratin materials, preferably for caring for keratin materials, in particular the skin of the body and/or of the face.

The invention also relates to a cosmetic process for making up and/or caring for keratin materials, in particular the skin and/or the lips, comprising at least one step of applying a composition as defined previously to said keratin materials.

Preferably, the invention also relates to a cosmetic process for caring for keratin materials, in particular the skin and/or the lips, comprising at least one step of applying a composition as defined above to said keratin materials.

In particular, a composition according to the invention may be used for combating the signs of skin aging.

Thus, the present application also relates to the use of a composition according to the invention for combating the signs of skin aging.

The composition may be applied to the skin by hand or using an applicator.

Throughout the description, including the claims, the expression “comprising a” should be understood as being synonymous with “comprising at least one”, unless otherwise specified. The expressions “between . . . and . . . ”, “comprises from . . . to . . . ”, “formed from . . . to . . . ” and “ranging from . . . to . . . ” should be understood as being inclusive of the limits, unless otherwise specified.

The invention is illustrated in greater detail by the examples presented below. Unless otherwise indicated, the amounts shown are expressed as mass percentages.

### EXAMPLE

### Measurement and Evaluation Methods

### Stability Measurement

The stability tests for the compositions were carried out in accordance with the guidelines imposed by standard ISO/TR 18811:2018. They are performed on formulations packaged in 30-ml opaque lacquered glass jars.

The measurements are carried out 24 hours and 1 month after formulation. They may be performed at three different temperatures, specifically at 20° C., at 4° C. and at 45° C.

### Percentage Degradation Measurement

The percentage degradation after storage is measured over 1 month (T=1 month), at 4° C. and at 45° C., by assaying the residual retinol in the composition after storage. The percentage degradation is measured by HPLC and calculated as a function of the theoretical initial percentage or the initial percentage measured by HPLC. The theoretical level corresponds to the theoretical initial retinol content.

### Measurement of the Organoleptic Properties of the Compositions

The appearance, color and odor are judged by a panel of three people, on formulations at T=24 hours for a jar at 20° C., at T=1 month for jars stored at 20° C., at 4° C. and at 45° C. If the composition has an odor said to be of “raw materials”, it can be disguised by fragrances, unlike an odor of oxidized oil (rancid odor).

### Measurement at 20° C.

The 30-ml opaque glass jars are stored at room temperature (20° C.) in individual cupboards.

### Measurement at 4° C.

The formulations in 30-ml opaque glass jars at 4° C. are stored at 4° C. in a Liebbern Gastro Line refrigerator supplied by the company Manumesure.

### Measurement at 45° C.

Oven storage takes place at 45° C. for 1 month using a Jouvan oven supplied by the company Manumesure. The 30-ml opaque jars are placed in the oven and left for the required time.

### Evaluation of the Colorimetric Properties

The colorimetric properties of the compositions were evaluated according to the following protocol.

The color of the compositions was evaluated after preparing 50-μm thin films on a contrast card, using a film spreader.

The colorimetric measurements were then taken using a Minolta CM2600D spectrocolorimeter at two points on the film.

The results are expressed in the (L*, a*, b*) system, in which L* represents the lightness, a* represents the red-green axis (−a*=green, +a*=red) and b* represents the yellow-blue axis (−b*=blue, +b*=yellow). Thus, a* and b* express the hue of the compound.

For each formulation, the color was measured at t=0 at 20° C., and then again at t=1 month (after storage at 45° C.).

The color difference ΔE* was then measured for each composition according to the following formula:

(ΔE*)²=(ΔL*)²+(Δa*)²+(Δb*)²

ΔL*=L* formulations at t=0−L* formulations at t=1 month

Δa*=a* formulations at t=0−a* formulations at t=1 month

Δb*=b* formulations at t=0−b* formulations at t=1 month

The higher the value of ΔE*, the greater the color change after 1 month.

### Example 1

Compositions I1 to I5 according to the invention and composition CC1 outside the invention are prepared using the weight proportions as detailed in the table below. The values are expressed as weight percentages relative to the total weight of the composition.

TABLE 1

| | Compounds | | | | | | |
|---|---|---|---|---|---|---|---|
| Phase | (INCI name) | I1 | I2 | I3 | I4: | I5 | CC1 |
| Phase A | Water | qs 100 | qs 100 | qs 100 | qs 100 | qs 100 | qs 100 |
| | Propanediol (Zemea | 3.0 | 3.0 | 3 | 3.0 | 3.0 | 3.0 |
| | Propanediol ® from Dupont | | | | | | |
| | tate and Lyle Bio Products) | | | | | | |
| | Glycerin (BP Glycerine | 8.0 | 8.0 | 8 | 8.0 | 8.0 | 8.0 |
| | from Organic Chemical | | | | | | |
| | Corporation) | | | | | | |
| | Trisodium ethylenediamine | 0.2 | 0.2 | 0.2 | 0.2 | 0.2 | — |
| | disuccinate (37% of active | | | | | | |
| | material) (Natrlquest ® E30 | | | | | | |
| | from Innospec Active | | | | | | |
| | Chemicals) | | | | | | |
| | Disodium EDTA | — | — | — | — | — | 0.15 |
| | (Dissolvine NA2-S from | | | | | | |
| | Azko Nobel from | | | | | | |
| | (Nouryon)) | | | | | | |
| | Adenosine (Adenosine | 0.1 | 0.1 | 0.1 | 0.1 | 0.1 | 0.1 |
| | Cosmetic Grade from | | | | | | |
| | Pharma Waldhof) | | | | | | |
| Phase B1 | Caprylyl glycol (Hydrolite | 0.5 | 0.5 | 0.35 | 0.5 | 0.5 | 0.5 |
| | CG from Symrise) | | | | | | |
| | Phenoxyethanol (GlysolV | 0.7 | 0.7 | 0.5 | 0.7 | 0.7 | 0.7 |
| | EPHL from Huntsman) | | | | | | |
| | Caprylic/capric triglyceride | 4.0 | 4.0 | — | 4.0 | 4.0 | 4.0 |
| | (DUB MCT 7030/MB from | | | | | | |
| | Stearinerie Dubois) | | | | | | |
| | Diisopropyl sebacate (Dub | 2.7 | 2.7 | 2.7 | 2.7 | 2.7 | 2.7 |
| | DIS from Stearinerie | | | | | | |
| | Dubois) | | | | | | |
| | Dicaprylyl carbonate | 2.0 | 2.0 | — | 2.0 | 2.0 | 2.0 |
| | (Cetiol CC from BASF) | | | | | | |
| | Sorbitan Tristearate (SP | 0.9 | 0.9 | 0.9 | 0.9 | 0.9 | 0.9 |
| | Span 65 MBAL-PW-(MV) | | | | | | |
| | from Croda) | | | | | | |
| | Cetearyl alcohol (and) | 0.6 | 0.6 | — | 0.6 | 0.6 | 0.6 |
| | Cetearyl Glucoside | | | | | | |
| | (Montanov 68 from | | | | | | |
| | SEPPIC) | | | | | | |
| | PEG-40 stearate (Myrj S40- | 2.0 | 2.0 | 2 | 2.0 | 2.0 | 2.0 |
| | PA-(RB) from Croda) | | | | | | |
| | Butyl | 3.0 | 3.0 | — | 3.0 | 3.0 | 3.0 |
| | methoxydibenzoylmethane | | | | | | |
| | (Avobenzone from MFCI) | | | | | | |
| | Octocrylene (Escalol 597 | 7.0 | 7.0 | — | 7.0 | 7.0 | 7.0 |
| | from ISP Ashland) | | | | | | |
| | Ethylhexyl salicylate | 5.0 | 5.0 | — | 5.0 | 5.0 | 5.0 |
| | (Chem OS from Chemspec | | | | | | |
| | Chemicals) | | | | | | |
| | Isohexadecane | | | 3 | | | |
| | Isononyl isononanoate | — | — | 5.5 | — | — | — |
| | Cetyl Alcohol | — | — | 2.5 | — | — | — |
| | Pentaerythrityl tetra-di-t- | 0.2 | 0.2 | 0.1 | 0.3 | 0.3 | — |
| | butyl | | | | | | |
| | hydroxyhydrocinnamate | | | | | | |
| | (Tinogard ® TT from BASF) | | | | | | |
| | BHT (Ionol CP from Oxiris | — | — | — | — | — | 0.2 |
| | Chemicals) | | | | | | |
| Phase B1 | Tocopherol (DL Alpha | 0.05 | 0.10 | / | 0.20 | 0.10 | / |
| | tocopherol from DSM | | | | | | |
| | Nutritional products) | | | | | | |
| Phase C | Polyacrylate crosspolymer- | 0.6 | 0.6 | — | 0.6 | 0.6 | 0.6 |
| | 6 (Sepimax Zen from | | | | | | |
| | SEPPIC) | | | | | | |
| | Carbomer (Carbopol Ultrez | 0.5 | 0.5 | | 0.5 | 0.5 | 0.5 |
| | 10 Polymer from Lubrizol) | | | | | | |
| | Ammonium | | | 1.2 | | | |
| | Polyacryloyldimethyl | | | | | | |
| | Taurate | | | | | | |
| | Sodium Acrylate/Sodium | | | 2 | | | |
| | Acryloyldimethyl Taurate | | | | | | |
| | copolymer & | | | | | | |
| | Isohexadecane & | | | | | | |
| | Polysorbate 80 | | | | | | |
| | Hydroxyethylcellulose | 0.1 | 0.1 | 0.1 | 0.1 | 0.1 | 0.1 |
| | (Natrosol 250 HHR CS | | | | | | |
| | from Ashland) | | | | | | |
| Phase D | Boron Nitride (Boron | 2.0 | 2.0 | — | 2.0 | 2.0 | 2.0 |
| | Nitride Powder BN PUHP | | | | | | |
| | 3002 from Saint Gobain | | | | | | |
| | Ceramics) | | | | | | |
| | Silica | | | 2 | | | |
| | Aluminum Starch | 2.0 | 2.0 | — | 2.0 | 2.0 | 2.0 |
| | Octenylsuccinate (Dry Flo | | | | | | |
| | Plus from Azko Nobel | | | | | | |
| | (Nouryon)) | | | | | | |
| D1 | Denatured alcohol | | | 3 | | | |
| Phase E | Sodium hydroxide (Caustic | 0.1754 | 0.1868 | 0.114 | 0.2096 | 0.1868 | 0.2 |
| | Lye from Gujarat Alkalies | | | | | | |
| | and Chemicals) | | | | | | |
| Phase D | Ascorbic acid (ACID | 0.05 | 0.10 | 0.50 | 0.20 | 0.10 | / |
| | ASCORBIC 80 MESH from | | | | | | |
| | BASF) | | | | | | |
| Phase E | Retinol (10% of active | 1 | 1 | 2 | 1 | 1 | 2.0 |
| | material) (Retinol 10SU | | | | | | |
| | from BASF) | | | | | | |

### Protocol for Preparing the Compositions

The ingredients of phase A are heated to a temperature of 70° C.-75° C. in a Minilab equipped with a planetary stirrer and an emulsifier.

The ingredients of phase B are heated to a temperature of 75° C. in a beaker by means of a hotplate and then poured into phase A.

The mixture is emulsified (emulsifier at 2000 rpm and planetary stirrer at 30 rpm) for 10 minutes.

Phase C is added and mixed (emulsifier at 3600 rpm and planetary stirrer at 40 rpm) for 10 minutes.

The heating system is stopped and the mixture is left to cool. Water is added.

The mixture is then pressurized to −0.60 Pa and the emulsifier is adjusted to 2400 rpm and the planetary stirrer to 50 rpm.

When the temperature of the mixture reaches 40° C., sodium hydroxide is added and the vacuum is adjusted to −0.40 Pa.

When the temperature of the mixture reaches 37° C., the fillers are added.

The speed of the emulsifier is increased to 2500 rpm and that of the planetary stirrer is reduced to 40 rpm while at the same time maintaining a vacuum at −0.60 Pa.

The other phases are added.

When the temperature of the mixture reaches 33° C., the Minilab is stopped and the formulation is transferred to a beaker.

The addition of retinol is carried out in a glovebox under an inert atmosphere (nitrogen). The appropriate amount of retinol is weighed and then added to the beaker, while stirring with a Rayneri at a speed of 650 to 700 rpm for 10 minutes.

The formulations are packaged, still under an inert atmosphere, in 30-ml glass jars and in 100-ml pill bottles, which are closed. The items are placed in an air lock and a vacuum cycle is performed to eliminate nitrogen. The packaged formulations are then recovered.

### Measurement of the Stability of Retinol in the Compositions

The results of the percentage degradation measurements for compositions I1 to I5 according to the invention and composition CC1 outside the invention are presented in table 2 below:

TABLE 2

| Composition | I1 | I2 | I3 | I4 | I5 | CC1 |
|---|---|---|---|---|---|---|
| T = 1 month at | 20% | 9% | 0% | 10% | 10% | 33% |
| 45° C. vs 4° C. | | | | | | |
| T = 1 month at | 20% | 0% | 0% | 10% | 10% | 40% |
| 45° C. vs | | | | | | |
| Theoretical | | | | | | |
| content | | | | | | |

Compositions I1 to I5 in accordance with the invention exhibit significantly lower retinol losses than that observed for composition CC1 outside the invention comprising EDTA and BHT, or not comprising the combination of di-t-butyl pentaerythrityl tetrahydroxycinnamate and ethylenediaminedisuccinic acid salt, ascorbic acid and/or tocopherol.

Thus, the results demonstrate that the compositions according to the invention are more stable than the compositions outside the invention.

1. A composition comprising at least:
retinol;
di-t-butyl pentaerythrityl tetrahydroxycinnamate; and
an ethylenediaminedisuccinic acid salt; and
at least one compound chosen from ascorbic acid or an analog thereof, and/or tocopherol or a derivative thereof.

2. The composition as claimed claim 1, comprising from 0.02% to 5.0% by weight of retinol, relative to the total weight of the composition.

3. The composition as claimed in claim 1, comprising from 0.01% to 2.5% by weight of di-t-butyl pentaerythrityl tetrahydroxycinnamate, relative to the total weight of the composition.

4. The composition as claimed in claim 1, comprising from 0.01% to 2.5% by weight of ethylenediaminedisuccinic acid salt, relative to the total weight of the composition.

5. The composition as claimed in claim 1, comprising from 0.05% to 0.50% by weight of ascorbic acid or an analog thereof, relative to the total weight of the composition.

6. The composition as claimed in claim 1, comprising from 0.01% to 1% by weight of tocopherol or a derivative thereof, relative to the total weight of the composition.

7. The composition as claimed in claim 1, wherein the ascorbic acid or analogs thereof are chosen from ascorbic acid, ascorbyl palmitate and magnesium ascorbyl phosphate.

8. The composition as claimed in claim 1, wherein the tocopherol and derivatives thereof are chosen from tocopheryl acetate, tocopheryl palmitate, tocopheryl linoleate, tocopheryl nicotinate, alpha-tocopherol, beta-tocopherol, delta-tocopherol and gamma-tocopherol, or isomers thereof.

9. The composition as claimed in claim 1, said composition comprises ascorbic acid or an analog thereof chosen from ascorbic acid, ascorbyl palmitate and magnesium ascorbyl phosphate.

10. The composition as claimed in claim 1, comprising ascorbic acid and tocopherol.

11. The composition as claimed in claim 1, wherein the ethylenediaminedisuccinic acid salt is chosen from the potassium, sodium and ammonium salts and the amine salts of ethylenediaminedisuccinic acid.

12. The composition as claimed in claim 1, wherein the mass ratio of di-t-butyl pentaerythrityl tetrahydroxycinnamate/ethylenediaminedisuccinic acid salt is between 0.1 and 3.

13. The composition as claimed in claim 1, which comprises less than 0.2% by weight of butyl hydroxytoluene (BHT) and/or ethylenediaminetetraacetic acid (EDTA) or a salt thereof.

14. The composition as claimed in claim 1, which also comprises at least one UV-screening agent.

15. The composition as claimed in claim 1, which also comprises at least one non-volatile hydrocarbon-based oil.

16. The composition as claimed in claim 1, which also comprises at least glycerol.

17. The composition as claimed in claim 1, which it is a cosmetic composition for caring for keratin materials.

18. A cosmetic process for making up and/or caring for keratin materials comprising at least one step of applying a composition as claimed in claim 1 to said keratin materials.

19. The composition as claimed in claim 2 comprising from 0.01% to 2.5% by weight of di-t-butyl pentaerythrityl tetrahydroxycinnamate, relative to the total weight of the composition; from 0.01% to 2.5% by weight of ethylenediaminedisuccinic acid salt, relative to the total weight of the composition, and from 0.05% to 0.50% by weight of ascorbic acid or an analog thereof, relative to the total weight of the composition.

20. The composition as claimed in claim 2 comprising from 0.01% to 2.5% by weight of di-t-butyl pentaerythrityl tetrahydroxycinnamate, relative to the total weight of the composition; from 0.01% to 2.5% by weight of ethylenediaminedisuccinic acid salt, relative to the total weight of the composition, and from 0.01% to 1% by weight of tocopherol or a derivative thereof, relative to the total weight of the composition.
